(19) United States
(12) Patent Application Publication
(10) Pub. No.: US 2022/0270767 A1
(43) Pub. Date: Aug. 25, 2022

(54) System that Determines and Reports Non-Medical Discharge Delays Using Standardized Patient Medical Information

(71) Applicant: Hospitalists Now, Inc., Austin, TX (US)

(72) Inventor: Merced Gonzales, Jr., San Antonio, TX (US)

(73) Assignee: Hospitalists Now, Inc., Austin, TX (US)

(21) Appl. No.: 17/738,202

(22) Filed: May 6, 2022

Related U.S. Application Data
(63) Continuation of application No. 16/447,806, filed on Jun. 20, 2019.
(60) Provisional application No. 62/829,555, filed on Apr. 4, 2019.

(51) Int. Cl. G16H 80/00; G06F 16/25; G16H 10/60

(52) U.S. Cl. CPC G16H 80/00; G16H 10/60; G06F 16/258

(57) ABSTRACT

A hardware processor-based patient system and method having an indexing and referential storage that collects, converts and consolidates patient information from various physicians and health-care providers and hospital facilities into a standardized format, including converting input medical information, files and treatment information provided by different sources and different formats into that standardized format, as well as specialized subprograms to detect avoidable days, which are delays in the discharge of a patient for non-medical reasons, as well as to collect information about avoidable days, which are delays in the discharge of a patient for non-medical reasons, and to transmit electronic communications to a hospital facility about any avoidable days, which are delays in the discharge of a patient for non-medical reasons so the hospital facility can take appropriate actions.

### RELATED APPLICATION DATA

This application is a continuation of U.S. patent application Ser. No. 16/447,806, filed Jun. 20, 2019, and claims the benefit of U.S. Provisional Application No. 62/829,555, filed Apr. 4, 2019, which is incorporated by reference into this utility patent application.

### TECHNICAL FIELD OF THE INVENTION

This invention relates to a system that determines and reports non-medical discharge delays using standardized patient medical information.

### BACKGROUND OF THE INVENTION

Medical records and medical treatment information are often saved in a specialized format on local data processor and storage servers at each particular hospital facility. Thus, while the medical records and medical treatment information in the same hospital facility may have a consistent format throughout that hospital facility, the medical records format used by other physicians and medical providers in other types of hospital facilities (e.g. long-term acute care hospital facility, a rehabilitation hospital facility, a skilled nursing hospital facility, or a terminal care hospice facility) is likely to be a non-standardized and inconsistent medical record format compared to the first hospital facility. That is, the medical records, files and treatment information at each of these different hospital facilities may be stored locally on a data processor and storage in a non-standardized format selected by whichever hardware or software platform is in use in the medical provider's local office. The inconsistency between stored medical record formats makes sharing medical records, files and treatment information between different hospital facilities quite problematic.

Further, patients being discharged and transferred to another particular type of hospital facility (e.g. long-term acute care hospital facility, a rehabilitation hospital facility, a skilled nursing hospital facility, or a terminal care hospice facility) will need to have their medical records, files and treatment information made available to physicians and medical providers in the new hospital facility. Patients being discharged and transferred home for home care and recuperation will likely need to share their medical records, files and treatment information files with other physicians and medical providers during follow-up visits to other recovery and rehabilitation facilities. And, patients being transferred to another unit in the same particular type of hospital facility will also need to have their medical records, files and treatment information made available to physicians and medical providers in the new unit in the hospital facility.

The length of a patient's hospital stay at a particular hospital facility will depend on the patient's condition and responsiveness to treatment. For every patient's ailment and condition, the patient's insurer will estimate a length of stay in the hospital facility for the purposes of insurance coverage. The patient's stay at the facility may, of course, be extended for medical reasons, such as, if there are delays in the patient's recovery due to post-surgical or other complications. If there are medical reasons for an extension of patient's stay at a particular hospital facility (e.g. a delay in the patient's discharge from the hospital facility), the expenses incurred during that extended stay are likely to be covered by the patient's insurer. That is, if the patient is not ready medically to be discharged from the hospital facility, the patient's insurer will continue to reimburse the facility and patient for costs associated with “medical reason” delayed discharge of the patient.

After treatment at a particular hospital facility has been completed and the patient is ready to be discharged from that hospital facility, the patient will be transferred to: (1) a long-term acute care hospital facility, (2) a rehabilitation hospital facility; (3) a skilled nursing hospital facility, (4) a terminal care hospice facility, (4) another type of hospital facility, (5) a different unit in the same particular type of hospital facility, or (6) the patient's home for home care and recovery. If, after treatment at a particular hospital facility has been completed and the patient is ready to be discharged from that hospital facility, there is a delay in the discharge of a patient for a non-medical reason, the costs associated with the extension of the patient's stay at the hospital facility after the patent is ready to be discharged are unlikely to be reimbursed by the patient's insurer. Such non-medical delays in the discharge of a patient (who is otherwise ready to be transferred out of the facility), are delays and associated costs that the hospital facility would like to avoid, sometimes called “avoidable days” or “delays.” Because the increased costs associated with the “avoidable days” or “delays” will not be reimbursed by the patient's insurance carrier, the hospital facility has an urgent need to determine, in real time, the reason for any non-medical delay in the patient's discharge from the hospital facility.

Because of delays in the discharge and transfer of patients, as well as the non-standardized medical records formats used by different hospital facilities, there is a significant difficulty for medical providers in different locations to anticipate when patients will arrive with certainty, and how a transferred patient's medical records, files and treatment information can be communicated to the new hospital facility using current patient information systems, due to the above challenges. Oftentimes, medical providers at new hospital facilities are given incomplete and out-of-date medical records, files and treatment information because such records are keep on separate, local area hardware processor-based data processor and storage systems, which cannot be readily-shared or consolidated due to format inconsistencies.

### SUMMARY OF THE INVENTION

Disclosed herein is a system for determining and reporting an avoidable days discharge delay event and a first facility causation for a patient that is ready for discharge from a first facility, the first facility causation is associated with the first facility operations or personnel and not related to the patient's clinical data or the patient's condition, the system having a hardware data processor coupled to a plurality of non-transitory storage devices and one or more input/output ports coupled to one or more input/output devices, said hardware data processor executes an avoidable days discharge delay subprogram. The non-transitory storage devices maintain a first set of facility data related to the first facility operations and personnel, and a first set of patient data including patient identification, patient treatment information, facility identification, and a discharge date for the patient. The input/output ports provide for management of data properties related to the first set of facility data stored in the non-transitory storage devices and the first set of patient data related to the patient admitted to first facility, the management of data properties includes conversion of patient data into a standardized data format and storage of patient data in the standardized data format.

In the disclosed system, upon modification of the patient's discharge date data to a later date for the patient that is ready for discharge from the first facility, the hardware data processor executes an avoidable days discharge delay subprogram to determine one or more first facility causations associated with the first facility operations or personnel and not related to the patient's clinical data or the patient's condition. The avoidable days discharge delay subprogram has the following functionalities: (a) accesses the first set of facility data stored in said non-transitory storage devices for the first facility where the patient is admitted, including data relating to the first facility operations and personnel, (b) analyzes said first set of facility data to identify the first facility causation correlated to the modification in the discharge date for the patient that is ready for discharge from the first facility, said analysis includes a review of said first set of facility data associated with the first facility operations and personnel without consideration of the patient's clinical data or the patient's condition, (c) determines if said first facility causation constitutes an avoidable days discharge delay event from the analysis of said first set of facility data without consideration of the patient's clinical data and the patient's condition, (d) generates one or more reports identifying the avoidable days discharge delay event and the first facility causation with a description of the reason for the discharge delay associated with first facility where the patient is admitted, said one or more reports being stored in said non-transitory memory storage; and, (e) transmitting and exporting through said input/output ports coupled to said hardware data processor said one or more reports identifying the avoidable days discharge delay event and the first facility causation to a predetermined group of personnel.

In the disclosed system, the first facility causation for the delay in the discharge date can be input based on a selection of causes from a menu. The first facility causation for the delay in the discharge date can be input based on a manual entry of a cause. The avoidable days discharge delay subprogram provides an on-screen alert under predetermined circumstances. The avoidable days discharge delay subprogram receives user input for the analysis of causation of the delay in the discharge date for the patient. The patient data includes one or more comments relating to the reasons for any delay in the discharge date. Reports are generated using a starting and ending date range, and the starting and ending date range is entered manually or selected from a pop-up calendar with selectable dates. The first set of facility data includes a facility name, facility type, and facility group related to the first facility. The facility name, facility type or facility group can be entered manually or selected from one or more lists of selectable facility names, facility types or facility groups.

The present invention is a hardware processor-based patient system and method having an indexing and referential storage that collects, converts and consolidates patient information from various physicians and health-care providers and hospital facilities into a standardized format, including converting input medical information, files and treatment information provided by different sources and different formats into that standardized format. Whenever the patient information is updated, it will first be converted into the standardized format and then stored in the collection of medical records on one or more of the hardware processor-based storage devices. After the updated information about the patient's condition has been stored in the collection, the content server, which is connected to the hardware processor-based storage devices, immediately generates a message containing the updated information about the patient's condition. This message is transmitted in a standardized format over the data processor and storage network to all physicians and health-care providers that have access to the patient's information (e.g., to a medical specialist to review the updated information about the patient's medical condition) so that all users can quickly be notified of any changes without having to manually look up or consolidate all of the providers' updates.

This present invention ensures that health care providers and hospital facilities are notified and have access to changes in the patient's status so they can readily adapt their own medical diagnostic and treatment strategy in accordance with other providers' actions. The message can be in the form of an email message, text message, or other type of message known in the art. The present invention system and method uses standardization of formatted patient information to enhance the performance and increases the efficiency of the present invention over known data processor and storage methods and systems through the storage of standardized formatted patient information from input medical information, files and treatment information in the hardware processor-based patient system and method and the use of an indexing and referential storage and specialized subprograms that uses hardware processor-based storage devices to collect and consolidate medical information, files and treatment information provided by different sources and different formats.

The present invention also uses the hardware processor-based patient system and method having an indexing and referential storage and specialized subprograms to detect avoidable days, which are delays in the discharge of a patient for non-medical reasons, as well as to collect information about avoidable days, which are delays in the discharge of a patient for non-medical reasons, and to transmit electronic communications to a hospital facility about any avoidable days, which are delays in the discharge of a patient for non-medical reasons so the hospital facility can take appropriate actions. The present invention generates real-time messages to transmit electronic communications to health care providers, facilities, and patients using the hardware processor-based patient system and method having an indexing and referential storage and specialized subprograms when a patient is ready to be discharged and transferred to a new hospital facility, and to identify the health care providers, facilities, and patients that will receive notices when a patient is ready to be discharged and transferred to a new hospital facility. The present invention data storage system and method enhances the performance and increases the efficiency of the present data processor and storage system using a hardware processor over known data processor and storage methods and systems by the use of an indexing and referential storage and specialized subprograms the detect delays (or avoidable days), generates/transmits notifications of non-medical delays in the discharge of a patient to hospital facilities and patients, and generates/transmits notifications of patient discharges to hospital facilities and patients.

This system supports continuity of care through enhanced communication and notification with relevant clinical and operational providers, and the ability to manage patient and treatment files and patient medical information using specialized subprograms and indexing and referential storage improves the efficiency and performance of hospital information systems by improving and integrating communications. Moreover, the system and method of the present invention allows a hospital facility to take appropriate actions to minimize any present or future delays in the discharge of patients from their hospital facility by detecting avoidable days, which are delays in the discharge of a patient for non-medical reasons, collecting information about avoidable days, which are delays in the discharge of a patient for non-medical reasons, and transmitting electronic communications to a hospital facility about any avoidable days, which are delays in the discharge of a patient for non-medical reasons so that facility can take appropriate actions.

The present invention is a specialized hardware processor-based system and method, which includes specialized data processor and storage readable medium and subprograms that are not available in a generic computer device, even though a user/provider accesses the system through a standard web browser on a computing device or client connected to the Internet or single or multi-tier network. The method provides a graphical user interface (GUI) by a content server, which is hardware or a combination of both hardware and software. A user, such as a health care provider or patient, can be given remote access through the GUI to view or update information about a patient's medical condition using the user's own local device (e.g., a personal data processor and storage or wireless handheld device). When a user wants to update the records, the user can input the update in any format used by the user's local device.

The present invention is a system comprising a hardware data processor coupled to a plurality of non-transitory storage devices and one or more input/output ports coupled to one or more input/output devices, said hardware data processor capable of execution of one or more subprograms, said hardware data processor receives through said input/output port patient treatment information describing a patient's medical condition and a patient's possible discharge date from one or more hospital facilities, said hardware data processor execution a first subprogram that converts said patient treatment information into a standardized data format using a hardware data processor coupled to a plurality of non-transitory storage devices, said hardware data processor stores said patient treatment information in said standardized format in one or more of said plurality of non-transitory storage devices; and said hardware data processor transmits an electronic communication through said input/output ports to one or more hospital facilities about said patient treatment information stored in said standardized format in one or more of said plurality of non-transitory storage devices.

Moreover, the hardware data processor makes a first delay determination by execution of a second subprogram if there is going to be a delay for non-medical reasons in the discharge of said patient compared to the possible discharge date shown in the patient treatment information stored in said one or more of said plurality of non-transitory storage devices; said hardware data processor transmits an electronic communication over said input/output ports to one or more hospital facilities about the first delay determination for non-medical reasons in the discharge of said patient compared to the possible discharge date shown in the patient treatment information stored in said one or more of said plurality of non-transitory storage devices including a description of the reason for said non-medical delay in the discharge of said patient;

Additionally, the input/output port provides remote access to said patient treatment information via a graphical user interface coupled to said hardware data processor that is coupled to one or more of said plurality of non-transitory storage devices so that said patient treatment information can be accessed and modified into a modified patient treatment information that reflects updated patient medical condition and an updated possible patient discharge date; and, said hardware data processor coupled to said one or more of said plurality of non-transitory storage devices makes a first data modification determination by execution of a third subprogram if said modified patient treatment information is different from the patient treatment information stored in said plurality of non-transitory storage devices.

Further, the hardware data processor executes a fourth subprogram that converts said modified patient treatment information into said standardized data format using said hardware data processor coupled to said plurality of non-transitory storage devices if, as determined by said hardware data processor, that there is a sufficient difference in the modified patient information compared to the patient information stored in said plurality of non-transitory storage devices; and the hardware data processor stores said modified patient treatment information in said standardized format in one or more of said plurality of non-transitory storage devices using said hardware data processor if, as determined by said hardware data processor, that there is a sufficient difference in the modified patient information compared to the patient information stored in said plurality of non-transitory storage devices, and the hardware data processor transmits an electronic communication through said input/output ports to one or more hospital facilities about said modified patient treatment information stored in said standardized format in one or more of said plurality of non-transitory storage devices.

The hardware data processor makes a modified delay determination by execution of a fifth subprogram if there is going to be a delay for non-medical reasons in the discharge of said patient compared to the updated possible discharge date shown in the modified patient treatment information stored in said one or more of said plurality of non-transitory storage devices; and, said hardware data processor transmits an electronic communication through said input/output ports to one or more hospital facilities about the delay for non-medical reasons in the discharge of said patient compared to the updated possible discharge date shown in the modified patient treatment information stored in said one or more of said plurality of non-transitory storage devices including a description of the reason for said non-medical delay in the discharge of said patient.

Compliance with 35 USC § 101 (Post-Alice) Analysis

The present invention complies with the 35 USC § 101 (utility) requirements for patentable subject matter because it is a system, but even if evaluated under the Alice analysis, the subject matter should be deemed patentable under the current multi-step analysis. Under Step 1 of the USPTO statutory subject matter analysis (post-Alice), it is shown that the present invention satisfies the “Statutory Category” requirement because the claimed invention is an improved systems and method that recites components or a series of steps.

Under Step 2A of Prong 2 of the statutory subject matter analysis (post-Alice), the present invention is not simply a “judicial exception” under 35 USC § 101 because the claimed invention is directed to enhancing the performance and efficiency of a data processor and storage system and network through the conversion and storage of standardized formatted patient information from input medical information, files and treatment information in the hardware processor-based patient system and method and the use of an indexing and referential storage and specialized subprograms that uses hardware processor-based storage devices to collect and consolidate medical information, files and treatment information provided by different sources and different formats. Moreover, the present invention uses an indexing and referential storage and specialized subprograms to detect delays (or avoidable days), generates/transmits notifications of non-medical delays in the discharge of a patient to hospital facilities and patients, and generates/transmits notifications of patient discharges to hospital facilities and patients. Because the present invention is not an abstract concept, a fundamental economic practice, a method of organizing human activity, an idea (standing alone), or a mathematical relationship, the present invention is not an abstract idea and is not simply a “judicial exception” under Step 2A of Prong 2 of the statutory subject matter 35 USC § 101 analysis (post-Alice).

Even if one were to examine whether Step 2B of Prong 2 of the statutory subject matter analysis (post-Alice) were satisfied (which is not necessary based on the above), the present invention is complies with the “Integrated into a Practical Application” requirement under the 35 USC § 101 analysis (post-Alice) because the claimed invention recites a combination of additional elements including storing information in a centralized repository server, providing remote access over a network to a centralized web-based server, converting updated patient information, medical files and treatment information that was input by a user in a non-standardized form into a standardized format, automatically generating a message about the location of updated information storage, transmitting the “real-time” messages to others, allowing users to access patients' medical records in the standardized format, and determining delays in the discharge of a patient for non-medical reasons with the collection, analysis and notification of a non-medical delay in the patent discharge to one or more hospital facilities, as well as detecting avoidable days, which are delays in the discharge of a patient for non-medical reasons, collecting information about avoidable days, which are delays in the discharge of a patient for non-medical reasons, and transmitting electronic communications to a hospital facility about any avoidable days, which are delays in the discharge of a patient for non-medical reasons so the hospital facility can take appropriate actions to minimize any present or future delays in the discharge of patients from their hospital facility by detecting avoidable days, which are delays in the discharge of a patient for non-medical reasons.

With these above-identified “additional elements,” the claimed invention as a whole integrates the system into a practical application (e.g. Example 41 and 42 from PTAB Subject Matter Eligibility Examples, p. 14-20); and, specifically, the additional elements set forth above recite specific improvements over prior art systems and methods by allowing users to share information in real time in a standardized format regardless of the format in which the information was input by the user and providing a hospital facility notice about any avoidable days, which are delays in the discharge of a patient for non-medical reasons so the hospital facility can take appropriate actions to minimize any present or future delays in the discharge of patients from their hospital facility by detecting avoidable days, which are delays in the discharge of a patient for non-medical reasons.

Thus, the claim is eligible because it is not directed to the recited judicial exception of an abstract idea. As noted previously, the claim as a whole does not merely describe generally a method of organizing human activity. But, even when viewed as a whole, the claim adds significantly more (i.e., an inventive concept) to any generalized method. The claimed invention as a whole does not merely describe how to generally “apply” the concept of storing and updating patient information in a data processor and storage environment; and, the claimed data processor and storage components are not recited at a high level of generality; and, the claimed components are not merely invoked as tools to perform an existing medical records update process. Accordingly, this invention is not simply implementing an abstract idea on a generic computer, but even if viewed as a generalized method, the claimed invention includes additional elements that make the claimed invention a practical application. For these reasons, present invention complies with the 35 USC § 101 (utility) requirements for patentable subject matter under the current multi-step analysis.

### BRIEF DESCRIPTION OF THE DRAWINGS

The objects and features of the invention will become more readily understood from the following detailed description and appended claims when read in conjunction with the accompanying drawings in which like numerals represent like elements and in which:

FIG. 1 shows a view of a system for hospital data in accordance with the present invention;

FIG. 2 shows a view of system data architecture useful for the present invention;

FIG. 3A shows an example of the quality panel for a selected patient;

FIG. 3B shows a detail of the quality panel and the action button for Avoidable Days;

FIG. 4A shows the Avoidable Days display screen;

FIG. 4B shows the action button for creating a new Avoidable Days entry;

FIG. 5A shows the Avoidable Days data entry screen;

FIG. 5B shows the Avoidable Days data entry screen with dropdown menu for selecting the reason for a delay;

FIG. 5C shows the Avoidable Days data entry screen with an entry in the Comment field; and

FIG. 6 shows the Avoidable Days display screen with the completed entry.

FIG. 7A shows the Reporting Dashboard for initiating an Avoidable Days report.

FIG. 7B shows the data selection screen for generating an Avoidable Days report.

FIG. 8 shows an example Avoidable Days report sorted by reason.

FIG. 9A shows a table of Avoidable Days reasons for a specified period.

FIG. 9B shows a bar chart representation of the avoidable Days reasons shown in FIG. 9A.

The objects and features of the invention will become more readily understood from the following detailed description and appended claims when read in conjunction with the accompanying drawings in which like numerals represent like element.

### DETAILED DESCRIPTION OF THE PREFERRED EMBODIMENTS

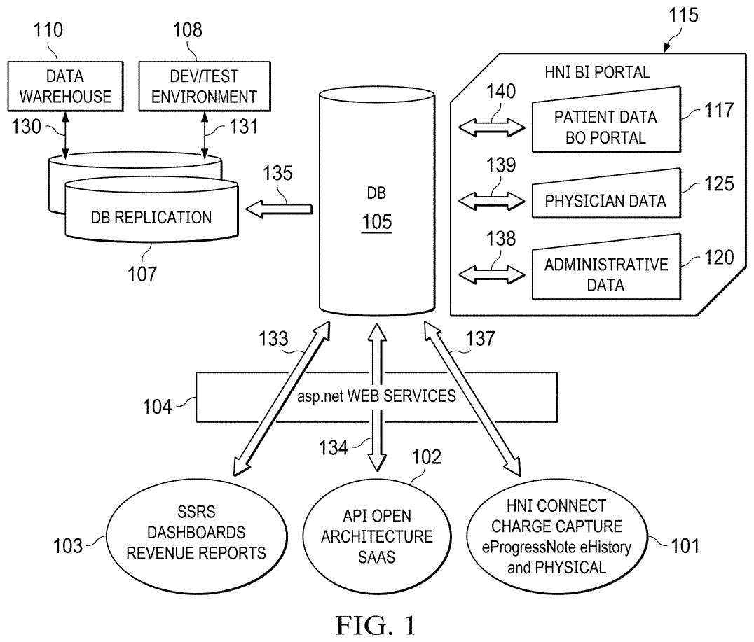

FIG. 1 shows the specialized architecture and storage connections and interaction used in the present invention. The primary storage 105 is coupled to a replication storage 107 through communication link 135. Replication storage 107 is coupled to the data warehouse 110 through communication link 130, and replication storage 107 is coupled to development/test environment protocols 108 through communication link 131.

The primary storage 105 is coupled to SQL Server Reporting Services (SSRS) services protocols 103 through communication link 133 through asp.net webservices support 104. The SSRS services protocols 103 support the “dashboards” and “revenue report” functions. The primary storage 105 is coupled to Application Programing Interface (API) services protocols 102 through communication link 134 through asp.net webservices support 104. The API services protocols 102 support the communication with open architecture protocols and Software as a Service (SAAS) protocols.

The present invention uses the hardware processor-based patient system and method shown in FIG. 1, which has an indexing and referential storage that collects, converts and consolidates patient information from various physicians and health-care providers and hospital facilities into a standardized format, including converting input medical information, files and treatment information provided by different sources and different formats into that standardized format. Whenever the patient information is updated, it will first be converted into the standardized format and then stored in the collection of medical records on one or more of the hardware processor-based storage devices. After the updated information about the patient's condition has been stored in the collection, the content server, which is connected to the hardware processor-based storage devices, immediately generates a message containing the updated information about the patient's condition. This message is transmitted in a standardized format over the data processor and storage network to all physicians and health-care providers that have access to the patient's information (e.g., to a medical specialist to review the updated information about the patient's medical condition) so that all users can quickly be notified of any changes without having to manually look up or consolidate all of the providers' updates.

This present invention ensures that health care providers and hospital facilities are notified and have access to changes in the patient's status so they can readily adapt their own medical diagnostic and treatment strategy in accordance with other providers' actions. The message can be in the form of an email message, text message, or other type of message known in the art. The file data storage system and method, as well as the standardization of formatted patient information, in the present invention enhances the performance and increases the efficiency of the present invention over known data processor and storage methods and systems through the storage of standardized formatted patient information from input medical information, files and treatment information in the hardware processor-based patient system and method and the use of an indexing and referential storage and specialized subprograms that uses hardware processor-based storage devices to collect and consolidate medical information, files and treatment information provided by different sources and different formats.

The API shown in FIG. 1 is initialized and runs a specialized program periodically to receive information from a hospital or customer facility. For example, the API code can be executed every 30 minutes to check if any new data has been received from a hospital or customer facility. If data has been received during that period of time, the API program will accumulate the received data and push it into the proper storage entries associated with the facility that transferred the data to the data processor and storage software used in the present system. Alternatively, the API subprogram on the system may reach out to certain hospital or customer facilities that provide access to their storage system so that the API subprogram can capture data from the hospital or customer facility data processor and storage system for uploading to the data to the data processor and storage software used in the present system.

The present invention shown in FIG. 1 is a specialized system and method, which includes specialized data processor and storage readable medium and subprograms that are not available in a generic computer device, even though a user/provider accesses the system through a standard web browser on a computing device or client connected to the Internet or single or multi-tier network. Also, the present invention works with multiple hospital information systems (HIS), Electronic Medical Record (EMR) systems, administrative data systems, and financial accounting systems. The method provides a graphical user interface (GUI) by a content server, which is hardware or a combination of both hardware and software. A user, such as a health care provider or patient, can be given remote access through the GUI to view or update information about a patient's medical condition using the user's own local device (e.g., a personal data processor and storage or wireless handheld device). When a user wants to update the records, the user can input the update in any format used by the user's local device.

As shown in FIG. 1, the data uploaded onto the storage 105 is formatted in a normalized manner with baseline data fields that include: visit number (encounter number), medical record number, patient name, diagnosis codes, gender (male/female), age (DOB), admission date, assigned doctor, location/department of facility patient admitted to. The demographics data for the patient is also placed in a normalized format of data fields that include: name of patient (first, middle, last name), visit number, medical record number, date of admission, contact address (home, permanent work or work addresses), insurance information (primary insurer: Medicare, Medicaid, BC/BS, secondary insurer: AFLAC, AARP, tertiary insurer: self, employer), parent/guardian information (if patient is minor), social security number (guarantor and patient). The insurance demographic information includes the policy number, group number, and insurance address for each insurer. Upon admission the baseline and demographic information for that patient is input into the storage 105, and the patient information may be accessed from, or input into, the data processor and storage system using a desktop data processor and storage device, mobile phone, intelligent pad devices, or other personal communication device.

The primary storage 105 of FIG. 1 is coupled to services protocols 101 through communication link 137 through asp.net web services support 104. The services protocols 101 support the invention services for Charge Capture, eProgress Notes, and eHistory & Physical functional protocols. The primary storage 105 is coupled to three portals in the HNI B1 Portal 115, with the primary storage 105 being coupled to Patient Data BO Portal 117 through communication link 140, and the primary storage 105 being coupled to Physician Data 125 protocols through communication link 139, and the primary storage 105 also being coupled to Administrative Data protocol 120 through communication link 138.

Whenever the patient information is updated, it will first be converted into the standardized format and then stored in the collection of medical records on one or more of the hardware processor-based storage devices. After the updated information about the patient's condition has been stored in the collection, the content server, which is connected to the hardware processor-based storage devices, immediately generates a message containing the updated information about the patient's condition. This message is transmitted in a standardized format over the data processor and storage network to all physicians and health-care providers that have access to the patient's information (e.g., to a medical specialist to review the updated information about the patient's medical condition) so that all users can quickly be notified of any changes without having to manually look up or consolidate all of the providers' updates. This ensures that each of a group of health care providers is always given immediate notice and access to changes so they can readily adapt their own medical diagnostic and treatment strategy in accordance with other providers' actions. The message can be in the form of an email message, text message, or other type of message known in the art.

The present invention shown also uses the hardware processor-based patient system and method having an indexing and referential storage and specialized subprograms to detect avoidable days, which are delays in the discharge of a patient for non-medical reasons, as well as to collect information about avoidable days, which are delays in the discharge of a patient for non-medical reasons, and to transmit electronic communications to a hospital facility about any avoidable days, which are delays in the discharge of a patient for non-medical reasons so the hospital facility can take appropriate actions. The present invention generates real-time messages to transmit electronic communications to health care providers, facilities, and patients using the hardware processor-based patient system and method having an indexing and referential storage and specialized subprograms when a patient is ready to be discharged and transferred to a new hospital facility, and to identify the health care providers, facilities, and patients that will receive notices when a patient is ready to be discharged and transferred to a new hospital facility. The present invention data storage system and method enhances the performance and increases the efficiency of the present data processor and storage system network over known data processor and storage methods and systems by the use of an indexing and referential storage and specialized subprograms to detect delays (or avoidable days), generates/transmits notifications of non-medical delays in the discharge of a patient to hospital facilities and patients, and generates/transmits notifications of patient discharges to hospital facilities and patients.

The present invention stores data in a more efficient and effective manner than previously used in other data storage systems through the use of an enhanced performance data storage sub-system using a self-referential, indexed data storage protocol and procedure that store all entity types in a single table after indexing is performed to prevent the creation of duplicative data entries in the data storage sub-system. The indexing protocols and procedures used in the enhanced data storage sub-system of the present invention reviews input data (received in health level 7 or HL7 format), and, before the creation of a new record in the data storage sub-system, the enhanced data storage sub-system of present invention using a self-referential, indexed data storage protocol and procedure searches existing records in the data storage sub-system to determine if the patient whose data is being reviewed was previously admitted into a particular hospital facility, a surrounding hospital facility, or any other related hospital facility connected to the present invention system.

If the enhanced data storage sub-system of present invention using a self-referential, indexed data storage protocol and procedure determines that the patient's data being reviewed relates to a patient that was previously admitted into a particular hospital facility, a surrounding hospital facility, or any other related hospital facility connected to the present invention system, then no new record for the patient is created in the data storage system and the input data is directed to the previously created record for that patient. If the enhanced data storage sub-system of the present invention using a self-referential, indexed data storage protocol and procedure determines that the patient's data being reviewed does not relate to a patient that was previously admitted into a particular hospital facility, a surrounding hospital facility, or any other related hospital facility connected to the present invention system, then a new record for the patient is created in the data storage system and the input data is directed to this new record for that patient.

A new record for a patient is only created when it is necessary, and when that patient has not been previously admitted into a particular hospital facility, a surrounding hospital facility, or any other related hospital facility connected to the present invention system. If the new data received for a patient relates to a patient that was previously admitted into a particular hospital facility, a surrounding hospital facility, or any other related hospital facility connected to the present invention system, no new patient record is created and the input data is directed to the previously created records in the data storage system. The avoidance and elimination of duplicate records creation for patients that were previously admitted into a particular hospital facility, a surrounding hospital facility, or any other related hospital facility connected to the present invention system results in an greater efficiency and effectiveness in the storage of patient records than has been previously known in the prior art data storage systems.

The present invention's use of an enhanced performance data storage sub-system using a self-referential, indexed data storage protocol and procedure supports record storage in a table after indexing, which also allows for faster searching of data stored therein compared to other data storage systems. Moreover, the enhanced performance data storage sub-system using a self-referential, indexed data storage protocol and procedure in the present invention allows for more effective storage of data than other data storage systems, such as image and unstructured data storage. And, the enhanced performance data storage sub-system using a self-referential, indexed data storage protocol and procedure in the present invention provides for more flexibility in the configuration of the data and records stored therein over other data storage systems.

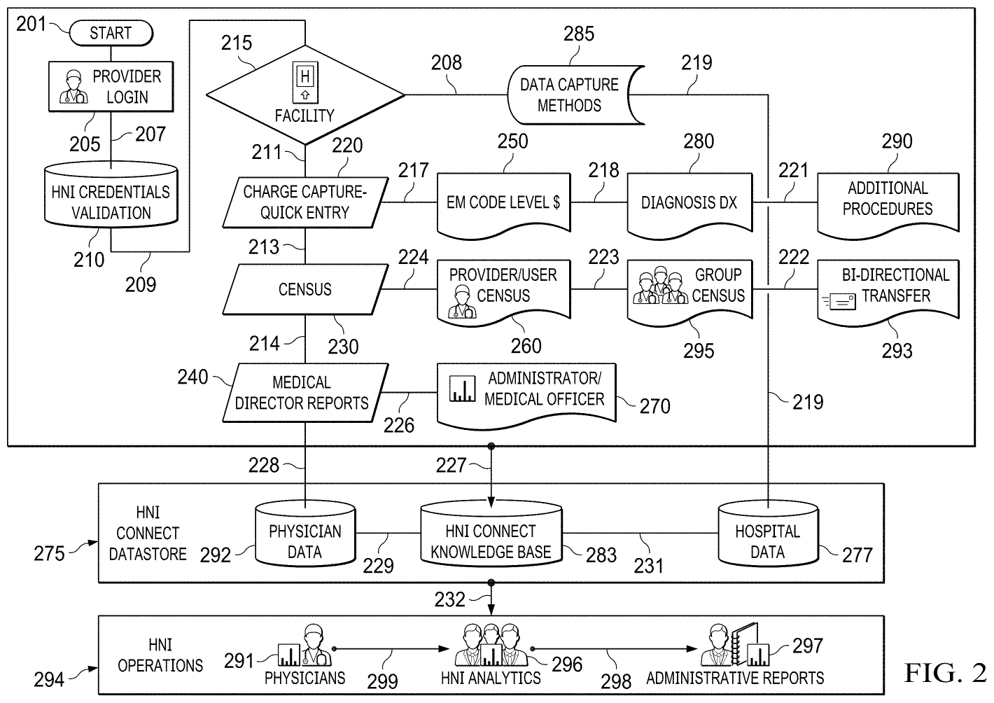

FIG. 2 shows the basic elements of the systems, how it interfaces with external data sources, such as the EMR and HIS systems and data elements associated with inpatient care. Through the present invention's data model, the system enables and integrates data from different types of systems in a seamless, flexible and fast manner. A software control program runs an on demand or by scheduler software routine to check a pre-defined directory that includes data either received or pulled down from different customer hospital systems coupled to the system. The software program starts by asking for a User Login, which must be correctly given to proceed in the program. After the User Login is given to the software program, the software program determines the User's rights and privileges in the next step of the software control program.

By rights and privileges, the type of information that defines such rights and privileges includes: (1) the type of user (e.g. doctor, biller, office manager, admin/executive, physical assistant/nurse practitioner, (2) facilities the User has privileges to (e.g. name of facilities, hospitals, etc. where User works or can access information about patients, hospitalists, assistants, etc.), (3) the type of facility that User works at or can access information about patients, hospitalists, assistants, etc. (hospital, long term care, nursing home, assisted living facility, rehabilitation facility, skilled nursing facility, etc.).

After the User's rights and privileges are determined, the software control program proceeds to the Home Screen 101 shown in FIG. 1 where the User can access several options, including the Census sub-program, Charge Capture sub-program, eProgress Notes sub-program, eHistory and Physical subprogram, the Changes sub-program, or the Statistics sub-program (SSRS). From the Home Screen 101 and if the User's rights and privileges permit, the User can access facility-based information including the following: (1) the type of facility that User works at or can access information about patients, hospitalists, assistants, etc. (hospital, long term care, nursing home, assisted living facility, rehabilitation facility, skilled nursing facility, etc.), (2) the identification of the User's patients such as patient name, room location, demographic information (age, primary address, etc.), (3) clinical information for each patient (primary diagnosis). This information may be accessed from, or input into, the data processor and storage system using a desktop data processor and storage device, mobile phone, intelligent pad devices, or other personal communication device.

From the Home Screen 101 and if the User's rights and privileges permit, the User may also input information regarding a patient such as the patient clinical history, diagnosis, treatment(s) received, medications (type and dosage), test results, x-rays or scan results, physical examination records, physician notes, lab results, prescription history. The patient prescription history would include drugs prescribed, dosages prescribed, and frequency of dosage, and this prescription history and present prescription types, amounts, and dosages can be shown graphically in the graphical formats shown in FIG. 16, and the software control program can analyze whether the current prescriptions for a particular patient are comparable to, greater than or less than a metric benchmark for similarly-situated patients.

The patient's physical and clinical history can be input or reviewed on-line using a Patient Data Portal 117, which is controlled by a Patient Data eHistory & Physical subprogram and specialized a graphical user interface (GUI). The patient information regarding the results of initial consults, clinical history reviews, and physicals are input into the data processor and storage system using the eHistory & Physical subprogram. Likewise, the progress of the patient can be monitored and updated by the hospitalist using an eProgress Note subprogram shown in the Home Screen 101 of FIG. 1. The information in the Patient Data Portal, Patient Data eHistory & Physical subprogram, and eProgress Note subprogram may be accessed from, or input into, the data processor and storage system using a desktop data processor and storage device, mobile phone, intelligent pad devices, or other personal communication device.

The eProgress Note subprogram will provide User feedback while a graphical user interface is completed with the patient progress information, with the feedback giving the doctor, physician, hospitalist or User other queries for information, suggestions on how to complete answers, suggested responses or lists of responses, or other relevant information. Other graphical user interface forms used in other subprograms can also provide the same type of User feedback when the User is providing responsive information to the system subprogram, such as the other queries for information, suggestions on how to complete answers, suggested responses or lists of responses, or other relevant information.

The User can also access the Statistics subprogram Dashboards, Survey Results and Revenue Reports screens shown in FIGS. 16 and 17 from the Home Screen 101 through the SSRS subprogram screen 103 shown in FIG. 1. The User can also input, modify and access patient information or physician information from the patient and physician subprograms pages, respectively. When the User is permitted to access information for a particular patient, the User can gage patient progress, physical information, treatment administered, or other patient information relating to test results, medication, eProgress Notes, and diagnosis.

The doctor, hospitalist or User can also access Statistics information that will allow him or her to gage their respective workloads compared to other doctors, hospitalists, or Users. The identity of other doctors, hospitalists, or Users may or may not be concealed or hidden from general access to all Users, but the doctor, hospitalist, or User can gage his workload against his co-workers to determine whether he or she is within standards for workload, behind or ahead of co-workers in terms of workload completed, or slower or faster than co-workers in terms of workload completion. The dashboards on FIG. 16 show bar charts, speedometer settings, line charts, and numerical tables so the User can judge his or her performance against a broader metric. By providing this comparative metric information to the User (e.g. comparing performance versus group of other users or other hospitalists), increases in worker productivity are possible, as well as highlighting areas where patient care can be enhanced through the identification of User or physicians that may not be expending sufficient time on particular cases or patients. In addition to gauging relative productivity, the User can also examine his or her individual performances to estimate the fees and wages that may be due to him or her for their work at the facility or with the patient.

All the physician or user information relating to a particular patient can be reviewed with the other physicians or users working with or treating the patient being identified to the User accessing the data processor and storage control system, as well as the pertinent information regarding patient progress, physical information, treatment administered, or other patient information relating to test results, medication, eProgress Notes, and diagnosis. The information relating to the Statistics, patient and physician subprograms may be accessed from, or input into, the data processor and storage system using a desktop data processor and storage device, mobile phone, intelligent pad devices, or other personal communication device.

Pre-defined specifications are shared with facility and then software program controls the additional method that takes into account the differences of the hospital data, normalizes to HNI specifications, and populates the appropriate tables and fields in the storage. This action occurs either “on demand” or with a scheduler. The workflow process begins as the census is applied to the application. The invention also includes a method to manage workflow for discharged patients. The present invention assimilates demographic, diagnosis code, charge codes, administrative, financial, and clinical data. This embodiment comprises a web-based, active server page (ASP) that provides real-time demographic, financial and clinical information. A user may access the system through any standard web browser operated on a computing device connected to the Internet or other network.

The system shown in FIG. 2 supports continuity of care through enhanced communication and notification with relevant clinical and operational providers, and the ability to manage patient and treatment files and patient medical information using specialized subprograms and indexing and referential storage improves the efficiency and performance of hospital information systems by improving and integrating communications. Moreover, the system and method of the present invention allows a hospital facility to take appropriate actions to minimize any present or future delays in the discharge of patients from their hospital facility by detecting avoidable days, which are delays in the discharge of a patient for non-medical reasons, collecting information about avoidable days, which are delays in the discharge of a patient for non-medical reasons, and transmitting electronic communications to a hospital facility about any avoidable days, which are delays in the discharge of a patient for non-medical reasons so that facility can take appropriate actions.

The present invention as shown in FIG. 2 is a specialized system and method, which includes specialized data processor and storage readable medium and subprograms that is not available in a generic computer device, even though a user/provider accesses the system through a standard web browser on a computing device or client connected to the Internet or single or multi-tier network. The method provides a graphical user interface (GUI) by a content server, which is hardware or a combination of both hardware and software. A user, such as a health care provider or patient, can be given remote access through the GUI to view or update information about a patient's medical condition using the user's own local device (e.g., a personal data processor and storage or wireless handheld device). When a user wants to update the records, the user can input the update in any format used by the user's local device.

The ability to manage patient and treatment files and information will improve physician communication and integrate hospital information systems. The system and method of the present invention allows a hospital facility to take appropriate actions to minimize any present or future delays in the discharge of patients from their hospital facility by detecting avoidable days, which are delays in the discharge of a patient for non-medical reasons, collecting information about avoidable days, which are delays in the discharge of a patient for non-medical reasons, and transmitting electronic communications to a hospital facility about any “non-medical” avoidable days, which are delays in the discharge of a patient for non-medical reasons so that facility can take appropriate actions. This system supports continuity of care through enhanced communication and notification with relevant clinical and operational providers.

The interactions of the specialized protocols in FIG. 2 begin at Start 201 that goes to the Provider Login 205. The Provider Login 205 proceeds to the HNI Credentials Validation 210 at step 207, which then proceeds to the Facility protocol 215 at step 209. The Facility protocol 215 can proceed to the Data Capture protocols 285 at step 208, which is coupled by connection 219 to the Hospital Data storage 277 located in the HNI Connect DataStore 275.

The Facility protocol 215 can also proceed to the Charge Capture-Quick Entry protocols 220 by step 211, which will proceed to the EM Code-Level protocol 250 by step 217, which will then proceed to the Diagnosis DX step 280 by step 218, which will then proceed to Additional Procedures protocols 290 by step 221. The Charge Capture-Quick Entry protocols 220 can proceed to the Census protocols 230 by step 213, which will then proceed to the Provider/User Census protocols 260 by step 224, which will then proceed to the Group Census 295 by step 223, which will then proceed to the Bi-Directional Transfer protocols 293 by step 222.

The Census protocols 230 can proceed to the Medical Director Reports protocols 240 by step 214, which is coupled by connection 228 to the Physician Data storage 292 located in the HNI Connect DataStore 275. The Medical Director Reports protocols 230 can also proceed to the Administration/Medical Officer protocols 270 by step 226, which is coupled by connection 227 to the HNI Connect Knowledge Data storage 283 located in the HNI Connect DataStore 275. The Physician Data storage 292 is coupled to the HNI Connect Knowledge Data storage 283 by connection 229, and the HNI Connect Knowledge Data storage 283 is connected to the Hospital Data storage 277 by connection 231.

The Census protocols 230 allow the User to access Provider/User Census 260 information, Group Census 295 information, or Bi-directional Transfer 293 information. Further, the data processor and storage control software supports the admission and discharge of patients using the Quick Entry protocols 220, including the ability to transfer patients to other facilities and the care of other physicians. When a User wishes to transfer a patient's care to another physician, the Bi-Directional Transfer 293 protocols or the Quick Entry 220 protocols may be used. After User Login, the User chooses the patient being transferred and puts in a description of why the transfer is occurring, the status of the patient, the identification of the new physician, hospitalist, or facility where the patient is being transferred to. The information relating to the patient transfer may be accessed from, or input into, the data processor and storage system using a desktop data processor and storage device, mobile phone, intelligent pad devices, or other personal communication device, and the physician, hospitalist, or User to whom the patient is being transferred will receive an email, text message or other notification about the transfer of patient care. That doctor, physician or hospitalist will be added to the User's having rights and privileges to that particular patient's information.

The storage 292, 283 and 277 in the HNI Connect Datastore 275 are coupled to the HNI Operations protocols 294, which include the Physicians protocols 291 connected by connection 299 to the HNI Analytics protocols 296, which is connected by connection 298 to the Administrative Reports protocols 297. The patented invention stores data in a more efficient and effective manner than previously used in other data storage systems through the use of an enhanced performance data storage sub-system using a self-referential, indexed data storage protocol and procedure that store all entity types in a single table after indexing is performed to prevent the creation of duplicative data entries in the data storage sub-system. The indexing protocols and procedures used in the enhanced data storage sub-system of the present invention reviews input data (received in health level 7 or HL7 format), and, before the creation of a new record in the data storage sub-system, the enhanced data storage sub-system of present invention using a self-referential, indexed data storage protocol and procedure searches existing records in the data storage sub-system to determine if the patient whose data is being reviewed was previously admitted into a particular hospital facility, a surrounding hospital facility, or any other related hospital facility connected to the present invention system.

If the enhanced data storage sub-system of present invention using a self-referential, indexed data storage protocol and procedure determines that the patient's data being reviewed relates to a patient that was previously admitted into a particular hospital facility, a surrounding hospital facility, or any other related hospital facility connected to the present invention system, then no new record for the patient is created in the data storage system and the input data is directed to the previously created record for that patient. If the enhanced data storage sub-system of the present invention using a self-referential, indexed data storage protocol and procedure determines that the patient's data being reviewed does not relate to a patient that was previously admitted into a particular hospital facility, a surrounding hospital facility, or any other related hospital facility connected to the present invention system, then a new record for the patient is created in the data storage system and the input data is directed to this new record for that patient.

A new record for a patient is only created when it is necessary, and when that patient has not been previously admitted into a particular hospital facility, a surrounding hospital facility, or any other related hospital facility connected to the present invention system. If the new data received for a patient relates to a patient that was previously admitted into a particular hospital facility, a surrounding hospital facility, or any other related hospital facility connected to the present invention system, no new patient record is created and the input data is directed to the previously created records in the data storage system. The avoidance and elimination of duplicate records creation for patients that were previously admitted into a particular hospital facility, a surrounding hospital facility, or any other related hospital facility connected to the present invention system results in an greater efficiency and effectiveness in the storage of patient records than has been previously known in the prior art data storage systems.

The present invention's use of an enhanced performance data storage sub-system using a self-referential, indexed data storage protocol and procedure supports record storage in a table after indexing, which also allows for faster searching of data stored therein compared to other data storage systems. Moreover, the enhanced performance data storage sub-system using a self-referential, indexed data storage protocol and procedure in the present invention allows for more effective storage of data than other data storage systems, such as image and unstructured data storage. And, the enhanced performance data storage sub-system using a self-referential, indexed data storage protocol and procedure in the present invention provides for more flexibility in the configuration of the data and records stored therein over other data storage systems.

Once a provider has logged into the information system and been validated for the system, an individual patient may be selected from the provider's census listing, and from the patient's data field, the Quality Panel screen can be accessed. The Quality Panel displays information related to measures that assess the cost of care, resources used to provide care, inappropriate use of resources, or efficiency of care delivered. Patient information on the Quality Panel is populated from the patient data input into the primary storage of the Hospital Information System.

The present system and method provide for identification, documentation and analysis of essential data for Avoidable Days, including documenting number of delayed days, identifying causes for delays and associated revenue losses for delayed days. Analysis of the causes for Avoidable Days allows hospitals the opportunity to remediate these causes thus decreasing loss of revenue related to Avoidable Days.

The term Avoidable Days (AD) is used to describe barriers that prolong patients' hospital stays when they are medically ready for discharge. Since Avoidable Days are days that a patient spends in a health care facility after being determined ready for discharge, but before actually being released either to home or to another health care setting, these Avoidable Days cause a loss in operational efficiency and account for a substantial loss of revenue for many health care organizations. Revenue losses related to Avoidable Days will likely increase as available funds become increasingly limited due to changes occurring in health care reimbursement. Accurate identification, documentation and analysis of causes is needed to reduce, eliminate or remediate Avoidable Days.

The length of a patient's hospital stay can also be extended for a number of non-medical related reasons. Non-medical related circumstances include when as a provider is ready to discharge a patient, but the requested facility (such as a skilled nursing center) does not have an available bed, the patient's family is refusing to allow discharge, transfer information has been delayed by an insurer, or a specialist physician consultation has been delayed. Delays that occur for non-medical reasons and result in additional days in the hospital are considered to be Avoidable Days, and the hospital is generally not reimbursed for these expenses.

The impact on health care availability and loss of revenue associated with these unreimbursed days is huge. When patients who are medically ready for discharge remain hospitalized, that reduces the availability of beds and resources for patients needing acute care. Delayed discharge also results in huge financial losses for the hospitals due to the unreimbursed expenses, which in turn, impacts the ability of those hospitals to operate in an effective and efficient manner.

The majority of patients admitted to hospitals have some form of medical coverage, either private medical insurance, or Medicare/Medicaid. Hospitals are reimbursed for medical care directly by the insurer. The present foundation for determining reimbursement to health care facilities is diagnosis-related groupings (DRGs). When a patient is admitted to a hospital, the reason for the admission is categorized by DRG codes that are assigned by the health care facility. These DRGs are based on the principal that patients with similar diagnoses would likely spend about the same amount of time as an inpatient and require similar resources while hospitalized. DRGs are how Medicare, Medicaid, and many other health insurance companies categorize hospitalization costs and determine how much to pay for a patient's hospital stay. Rather than paying the hospital for what was actually spent caring for a hospitalized patient, Medicare pays the hospital a fixed amount based on the patient's DRG or diagnosis code. The DRG classification system standardizes prospective payment to hospitals and encourages cost containment initiatives. A DRG payment is generally expected to cover all charges associated with an inpatient stay from patient admission to until discharge. The DRG also includes any services provided by an outside provider.

For DRGs, patients are categorized with respect to diagnosis, treatment, and length of hospital stay. DRGs are assigned based on a number of variables, including: principal diagnosis; secondary diagnosis(es); surgical procedures performed; co-morbidities and complications that may affect treatment (such as diabetes or pulmonary disease); age and sex of the patient; and discharge status.

The original intent of DRGs was to identify the “products” provided by a hospital, such as a procedure like an appendectomy. Since patients within each category or group are assumed to be clinically similar and are expected to use the same level of hospital resources, DRG payments are based on the care given to and resources used by a “typical” patient within the group. DRGs have been used in the U.S. since 1982 to determine how much Medicare pays hospitals for each such “product” it provides. While DRGs were originally used to determine standardized payments under Medicare, similar prospective payment systems are now utilized by many private insurers.

If a hospital treats a patient while spending less than the prospective DRG payment, it makes a profit. However, if a hospital spends more than the prospective DRG payment treating a patient, it has a loss. Medicare, Medicaid and most other insurers do not reimburse hospitals for days a patient spends in the hospital after the patient is deemed medically ready for discharge. These extra days in the hospital, deemed Avoidable Days, are one of the primary avenues through which hospitals lose revenue. Delayed discharges also have an impact on hospitals' ability to accommodate new patients and deliver healthcare effectively and efficiently. A system and method is needed to capture, analyze and report the reasons for Avoidable Days in order to reduce or eliminate delayed discharges to decrease revenue loss while increasing the overall hospital efficiency.

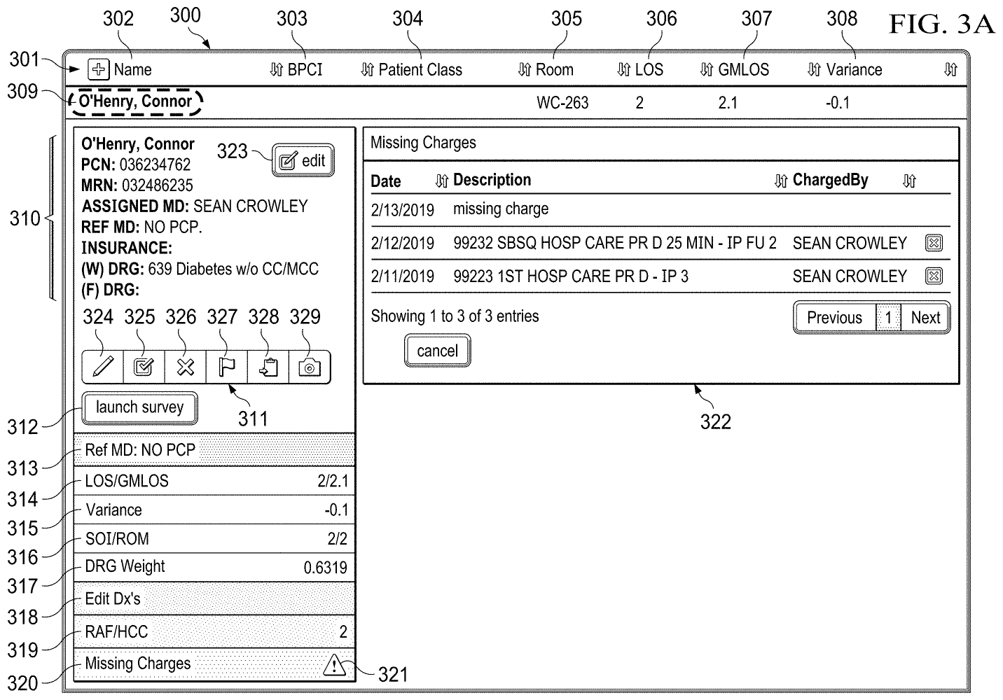

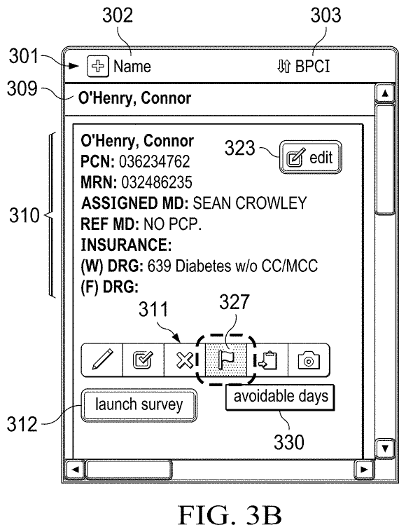

The Quality Panel screen 300 seen in FIG. 3A has a patient census bar 301 with headings for, optionally, patient name 302, BPCI (Bundled Payments for Care Improvements) 303, Patient Class (e.g., Inpatient, Outpatient, ER,) 304, Room number 305, LOS (Length of Stay) 306, GMLOS (Geometric Mean Length of Stay) 307, and Variance (difference between LOS and GMLOS) 308. The patient census display bar 309 shows details for the selected patient for each of the above patient census bar options. The Quality Panel screen displays a patient's current data overview 310, including, patient name, PCN (Primary Care Network), MRN (Medical Records Number), Assigned MD, Referring MD, Insurance, (W) DRG (working diagnosis related group), and (F) DRG (final diagnosis related group).

A number of action buttons are provided on the quality panel to allow for management of data. A launch survey action button 312 sends a customized survey for the selected patient to complete prior to discharge and an Edit action button 323 allows for editing of the Quality Panel data. A series of action buttons 311 allows the physician to add, manage or transfer data associated with the case. The pencil action button 324 functions to add physician notes on the case. The check-mark action button 325 allows the physician to electronically “check-out” of a case at the end of a shift. The X-mark action button 326 deletes that case from the physician's case listing. The flag action button 327 opens a data entry screen to report discharge delays that result in Avoidable Days. The clipboard action button 328 allows for a copy of the patient information to be sent electronically when a patient is transferred to another facility, such as a skilled nursing facility. The camera action button 329 takes a snapshot of the screen image that may be used for identification of a case. For example, the snapshot can be used in face-sheets associated with the case to give the physician an at-a-glance summary of the case.

The Quality Panel 300 has navigation bars that will display additional data in an adjacent window when actuated. Navigation bars include Referring MD (Primary Care Provider) 313, LOS/GMLOS (length of stay ratio) 314, Variance in LOS/GMLOS 315, SOI/ROM (Severity of Illness/Risk of Mortality) 316, DRG weight 317, Edit DX (edit diagnosis) 318, RAF/HCC (Risk Factor Adjustment/Hierarchal Condition Category) 319, and Missing Charges 320. Each navigation bar also displays the current data for that item.

On the Missing Charges bar 320, there is an alert icon 321 indicating that there are charges missing for the selected patient. In FIG. 3A, the Missing Charges navigation bar 320 has been activated, and the display window 321 shows a description for each hospital day for the selected patient, indicating the day with missing charges. The charges may be missing because they have not yet been entered, but often, missing charges indicate a delay in discharging a patient who is medically ready to go home or be transferred to another facility, and this delay results in an unreimbursed Avoidable Day.

Understanding the causes for Avoidable Day charges is key to decreasing the number of Avoidable Days and the accompanying loss of revenue for the unreimbursed days. The present system and method captures data related to the number of days along with the reasons for the delays, and this information provides a way to analyze and report data for the Avoidable Days, which in turn allows for remediation planning to prevent future revenue loss.

FIG. 3B is an enlargement of a portion of the Quality Panel in FIG. 3A and references the same elements seen in FIG. 3A. Hovering over the flag icon for the Avoidable Days action button 327 displays the words “avoidable days” 330, and actuating the button leads to an Avoidable Days display screen.

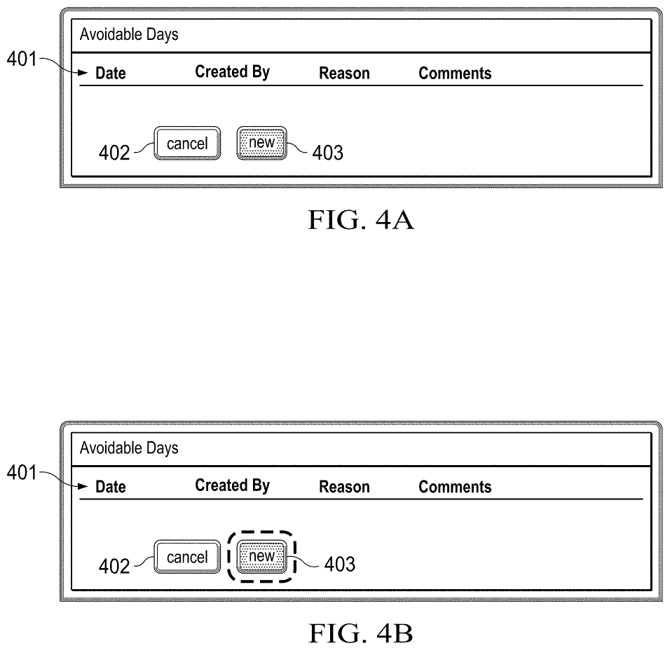

The Avoidable Days display screen shown in FIG. 4A is blank, indicating no entries have yet been input to the system. The header bar 401 shows headings for: date; created by; reason; and comments. There are action buttons for Cancel 402 to leave the page and New 403 to make a new Avoidable Days entry. In FIG. 4B the “New” action button 403 is highlighted and actuating the button opens an Avoidable Days entry screen.

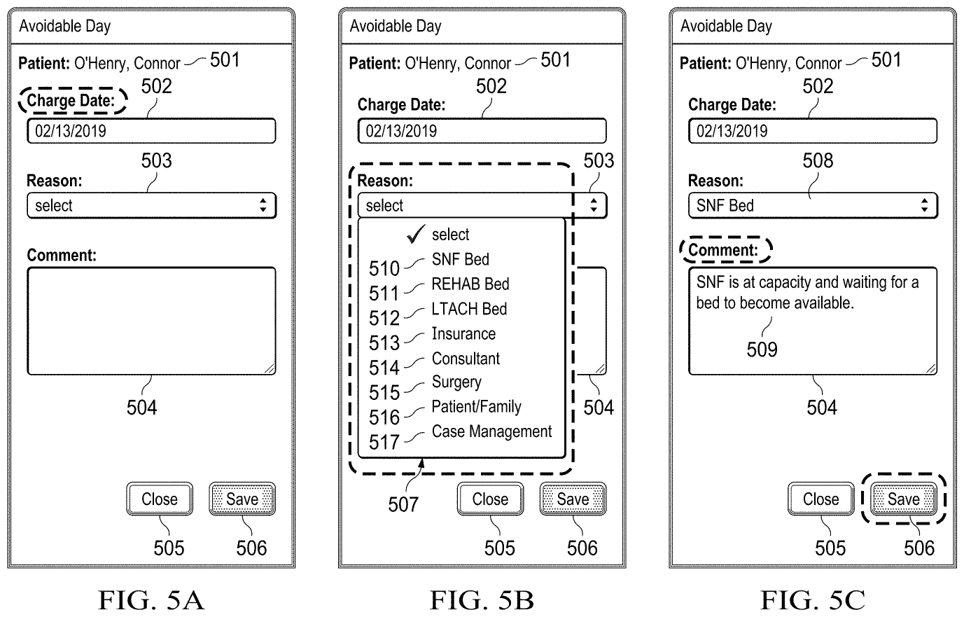

FIG. 5A shows the Avoidable Days entry screen displaying the patient name 501. The Charge Date entry field 502 shows the date with the missing charges. The Reason navigation bar 503 will open a dropdown menu of selectable reasons for the discharge delay. The Comments entry field 504 allows entry of additional information related to the selected reason. Action buttons will Close 505 the screen or Save 506 the changes.

The dropdown Reason menu 507 is shown FIG. 5B with a listing of selectable reasons. Selectable reasons include SNF Bed (Skilled Nursing Facility) 510, REHAB Bed (Rehabilitation Facility) 511, LTACH Bed (Long-Term Acute Care Hospital) 512, Insurance 513, Consultant 514, Surgery 515, Patient/Family 516, and Case Management 517.

There are a number of reasons why a discharge or transfer of a patient does not take place in a timely manner following completion of discharge orders or when the admitting physician has deemed the patient medically ready for discharge. Delays may be facility based, personnel based, or patient/family based. Facility based delays may occur if there are no beds available in the requested Skilled Nursing Facility, Rehabilitation Center, or Long-Term Acute Care Hospital. Personnel based delays may occur if consultants, insurers or case managers do not provide the required input in a timely manner. Patient/Family-based delays may occur if a patient has refused discharge, or if the family is not ready or willing for the patient to be discharged. Surgery based delays may occur if there are delays in scheduling or completing a surgical procedure, or if complications arise either before or after a procedure.

Facility Based Delays. A substantial number of patients, especially elderly patients or trauma patients, will require a transfer from an acute care hospital to another facility for rehabilitation or continued recovery. Some patients will only require a short stay in another facility before being ready to go home, but some of these patients will require transfer to a facility for a long rehabilitation period or potentially permanent long-term care. The type of care required, as well as the anticipated duration of residence, are factors that determine where to transfer a patient, and part of the discharge process is assessing these needs for a patient prior to discharge. Proximity to family may also be a factor considered in selecting a facility for a patient.

Depending on the care needs, a patient may be transferred to a Skilled Nursing Facility (SNF), a Rehabilitation facility (REHAB), or a Long-Term Acute Care Hospital (LTACH) and selections from the Reasons dropdown menu 507 for facility type delays include SNF Bed 510, REHAB Bed 511, and LTACH Bed 512. One cause of delayed discharge occurs when a desired facility does not have a bed immediately available. Not checking on availability or exploring alternative options in a timely manner can be a cause of Avoidable Days as much as the lack of space in a desired facility.

SNF Bed (Skilled Nursing Facility) 510. Skilled Nursing Facilities provide medically necessary professional services from nurses, physical and occupational therapists, speech pathologists and audiologists, and are a step-down in care needs from an acute care hospital. A SNF is typically required for elderly or disabled patients who are either not ready at the present time, or will not become able, for the rigors of an intensive rehabilitation setting. Avoidable Days may occur because a SNF bed is not immediately available when a patient is ready to be transferred. Patients being transferred to a SNF need more care than is available in an intensive rehabilitation facility or home health setting, and will, therefore, remain hospitalized until a bed in an appropriate facility becomes available. Because the patient often spends an extended period of time in a SNF, a facility that is located near the patient's family is preferable, and locating a suitable facility that is conveniently located for the family can be another cause of Avoidable Days.

REHAB Bed (Rehabilitation Facility) 511. Rehabilitation Facilities are inpatient facilities that provide intensive rehabilitative services. Rehabilitative services may be either acute or a sub-acute, depending on the amount and level of therapy required. Sub-acute rehabilitation facilities provide two or fewer hours per day of physical, occupational and speech therapy at a low level of intensity. Acute inpatient rehabilitation hospitals provide more hours of therapy per day, at a more intensive level than in a sub-acute setting. Stays in acute inpatient rehabilitation hospitals are generally much shorter than stays in sub-acute rehabilitation facilities, and whether the patient needs or can tolerate acute or sub-acute therapy will inform what type of facility is chosen for a patient transfer.

An inpatient acute rehabilitation facility will be able to provide more intensive rehabilitation than a skilled nursing facility or home-based rehabilitation service. Patients who are recovering from joint surgery, such as a knee replacement, usually need a period of intensive rehabilitation in order to regain mobility. Patients who have experienced a stroke or a traumatic brain injury usually required a longer rehabilitation period to relearn skills lost due to damage to the brain. Elderly or disabled patients may also require a longer period of time in a rehabilitation facility to regain strength after an extended hospital stay for an illness, even if there was no surgery. Discharge can be delayed if a REHAB bed in a facility with the appropriate level of intensity is not immediately available. Matching a patient's needs, such as mobility recovery or regaining strength, to an appropriate or convenient Rehabilitation Facility can also cause a delay in discharge leading to Avoidable Days in the hospital.

LTACH Bed (Long-Term Acute Care Hospital) 512. Long-Term Acute Care Hospitals specialize in treating patients with serious medical conditions that require extended hospitalization for acute care on an ongoing basis. Patients requiring a transfer to a LTACH have complex or multiple medical conditions requiring intensive or specialized care, especially if the patient has a medical device dependency. LTACHs are able to provide more services than are available in a skilled nursing or rehabilitation facility, but the patient no longer requires the critical care level of a short-stay acute care hospital. LTACH transfers are most commonly patients with progressive illnesses, medical device dependencies or traumatic injuries who may not progress to the mobility levels required for a rehabilitation setting, but require more specialized care than is provided in a skilled nursing center. Because of the long-term nature of the stay, locating an available bed in an LTACH or waiting for a bed in a facility near the family can cause delays in discharge leading to unreimbursed Avoidable Days.

The claimed System and Method for Capturing, Analyzing and Reporting Avoidable Days is a tool that allows hospitals to determine the causes of facility based Avoidable Days in order to take action for the reduction, elimination or remediation of these causes. The system identifies facility-based delays by patient, number of Avoidable Days and cause for the delay. Option buttons allow users to select an element (SNF Bed 510, REHAB Bed 511 or LTACH Bed 512) related to the cause. Details associated with the cause (bed was not available, the facility was at capacity, identification of the facility, etc.) can be entered in a comment section to provide additional information that can be used for analysis.

Once the causes have been identified, an analysis of the data can show patterns related to the causes that can be used to take action to reduce or eliminate the Avoidable Days. For example, if the analysis shows that it takes an average of only two days to locate a bed in a SNF but an average of five days to find a bed in a LTACH, options for LTACH placement should be researched much earlier than for SNF placement to avoid delays in discharge. If the analysis shows a particular facility is frequently at capacity causing transfer delays, efforts can be made to add equivalent facilities to the roster of providers to offer more placement options. Remediation options can also be planned based on the causal analysis. For example, a hospital might create a new unit within the hospital as a rehabilitation or step-down setting that provides reimbursable services.

The actions for reduction, elimination and remediation all serve to decrease revenue losses due to Avoidable Days. By understanding the causes of the Avoidable Days, the hospital is better able to reduce or eliminate Avoidable Days or the hospital may make remediation plans that lead to reimbursement.

Personnel Based Delays. Personnel based delays may occur if insurers, consultants or case managers do not provide the required input in a timely manner. Delays in insurance approval for transfers, delays in arranging facility transfers, surgical services delays and delays in signing-off on cases ready for discharge can cause Avoidable Days 503. Selections from the Reasons dropdown menu 507 for personnel type delays include Insurance 513, Consultant 514, Surgery 515, and Case management 517.

Insurance 513. Approval is required from the insurer for transfers to another facility, starting home health service, or providing durable medical supplies. If approval from the insurer for any of these types of post-discharge needs is delayed, it may cause Avoidable Days to occur while the transfers or services are scheduled or medical supplies are acquired. These Avoidable Days are generally not reimbursed even though the delay was on the part of the insurer.

Hospital Facilities can complain to the insurance company and/or renegotiate rates, or DRG steps can be reassessed. It is important to identify and document these types of insurance-based delays so the primary care physician or hospitalist is not held responsible for delays in transferring or discharging a patient.

Consultants 514. Delays may be caused by a specialist physician who has been consulted on a case. If the consultant does not sign-off on a case in a timely manner, the discharge may be delayed. If a case is referred over a weekend or holiday, the consultant may not complete the evaluation and sign-off on the discharge in a timely manner, or records may not be forwarded in a timely manner over a holiday or weekend. If labs or tests requested by the consultant are not ordered or completed in a timely manner, the results may not be received in time for the consultant to sign off on the case before the projected discharge. Delays may also occur if a specific specialist is requested who is unavailable at the time or who is unwilling or unable to complete a consultation prior to the projected discharge.

If another specialist is called in to complete an evaluation, a complaint may be registered by the original consultant if someone else is assigned. Identifying and documenting consultant delays affords support for assigning another consultant by providing an explanation for the new assignment, such as a no-show from the original consultant.

It is important to capture which consultants are responsible for Avoidable Days and document the delays. If a particular consultant is responsible for a high percentage of the consultant-based delays, the facility can transmit electronic communications to the consultant of the documented delays and work with the consultant to reduce the delays. The facility may also need to provide additional consultants in a specific discipline to improve turn-around times for discharge evaluations, or the facility may have to remove a specific consultant from the approved roster or reduce the number of cases assigned to that consultant. All of these measures can lead to reduction in the number of Avoidable Days, but the delays must first be identified, documented and analyzed in order to evaluate the causes of the discharge delays.

Surgery 515. Surgical services delays may be a cause of delayed discharge. Surgical services delays include: late arrivals, missing or incomplete paperwork, lack of available space in recovery, or low staffing in surgical departments. Late arrival of surgeons, anesthesiologists or other surgical staff may cause a surgical services delay, not just for a specific procedure, but may also cause a backlog for other scheduled procedures. Lack of available beds in recovery or other post-operative unit can delay a surgical procedure. Under-staffing in the surgical department for the scheduled procedures can cause surgical delays because the available staff are too busy to handle additional patients. Another common cause of surgical delays is missing or incomplete paperwork, such as informed consents or other forms requiring signatures.

Delays in surgical services can lead to Avoidable Days in the hospital. Many of the causes for surgical services delays are personnel-based and once the causes have been identified and analyzed, remediation of the causes through improved communication, scheduling, or staffing can decrease the delays that lead to Avoidable Days.

Discharge delays can also occur due to surgical complications. If complications arise, a patient may be moved to an Intensive Care Unit (ICU), Critical Care Unit (CCU), or Intermediate Care Unit (IMC) instead of being discharged. Slower than anticipated recovery, co-morbidities, or other complications, such as infections or drug reactions, can also cause a delay in discharge, however, these types of delays are more likely to be reimbursed and are less likely to result in unreimbursed Avoidable Days.

Case Management 517. Every patient is assigned a case manager to provide assistance within, between, and outside of the medical facility. Case managers work with patients, families and other professionals to make provision for the needs of the patient, including [symbol] transfers to another facility, such as a rehabilitation center. Discharge delays can occur if verification of insurance coverage for transfers is delayed, delays in case review, delays in signing-off on transfers, and delays in other phases of the discharge or transfer process.

The claimed System and Method for Capturing, Analyzing and Reporting Avoidable Days allows hospitals to determine the causes of personnel-based Avoidable Days in order to take action for the reduction, elimination or remediation of these causes. The system identifies personnel-based delays by patient, number of Avoidable Days and cause of delay. Options on the Reasons dropdown menu 507 allow users to select an element (Insurance 513, Consultant 514, Surgery 515 or Case Management 517) related to the cause. Details associated with the cause (insurance verification delayed, requested consultant not available, etc.) can be entered in the comment section to provide additional information that can be used for analysis.

Once the causes have been identified, an analysis can show patterns related to the causes that can be used to reduce or eliminate the Avoidable Days. For example, if the analysis shows that surgical procedures scheduled on Thursdays generate more surgical services delays, the Thursday schedules and staffing can be reviewed and changed, if needed. Or, if the analysis shows a particular insurance provider frequently delays approval of services, efforts can be made to communicate to the provider the need for timely responses. Remediation options can also be planned based on the causal analysis. For example, if discharges are highest for specific days or times, additional case managers may be needed for those times.

Reduction, elimination and remediation all serve to decrease revenue losses due to Avoidable Days. By understanding the causes of the Avoidable Days, the hospital is better able to reduce or eliminate Avoidable Days or the hospital may make remediation plans that lead to reimbursement.

Patient/Family Delays 516, Patient Delays. Patients sometimes refuse to be discharged, either to home or for a transfer to another facility, even though the physician has determined medical readiness. A patient who has been through a difficult illness or surgical procedure may not feel ready to go home and may refuse discharge from the hospital, particularly when the cause of the hospitalization has resulted in a life-altering change, such as an amputation. A patient may also refuse discharge if the specific rehabilitation or skilled nursing facility requested does not have an available bed, or if the patient doubts the ability of the new facility to meet the patient's needs. These patient-based delays result in Avoidable Days and may be a significant cause of lost revenue for a hospital, especially if the delays occur for an extended period.

Family Delays. A discharge can be delayed if the patient's family is unwilling, unable or unready to care for the patient. A discharge may be refused because the family feels inadequate or is unwilling to handle the care needs of the patient, such as a patient who now requires the use of a medical device, such as an ostomy pouch. Another reason a family may refuse discharge is that the family's home is not ready to receive the patient. For example, if durable medical equipment, such as a wheelchair, hospital bed, or oxygen generator, is required for a patient at home, discharge may be delayed until these items are available. If changes must to be made at the home to accommodate the patient's needs, such as installation of ramps or other assistive structures, the discharge may be delayed until the home is prepared to receive the patient.

The claimed System and Method for Capturing, Analyzing and Reporting Avoidable Days allows hospitals to determine the causes of Patient/Family-based Avoidable Days in order to take action for the reduction, elimination or remediation of these causes. The system identifies the delays by patient, number of Avoidable Days and cause of delays. There is an option on the Reasons dropdown menu 507 that allow users to select Patient/Family 516 delays as the cause of the Avoidable Days. Details associated with the cause (reason for refusal or delay) can be entered in the comment section to provide additional information that can be used for analysis.

Once the causes have been identified, an analysis can be used to reduce or eliminate the Avoidable Days. For example, if the analysis shows patients or families frequently feel unprepared for care tasks, efforts can be made to better educate patients and families about post-hospital care. For example, allowing a patient or family member to learn and practice care tasks while in the hospital can increase confidence in the ability to handle those care tasks that will be continued after discharge. If the cause of the delays is related to acquiring the durable medical equipment needed, efforts can be made to order the necessary equipment for delivery to either the hospital or the patient's home to coincide with anticipated discharged date. Remediation options can also be planned based on the causal analysis. For example, if delays are more likely to occur for the specific days and times when discharges are highest, additional case managers may be needed for those times to facilitate the discharge process.

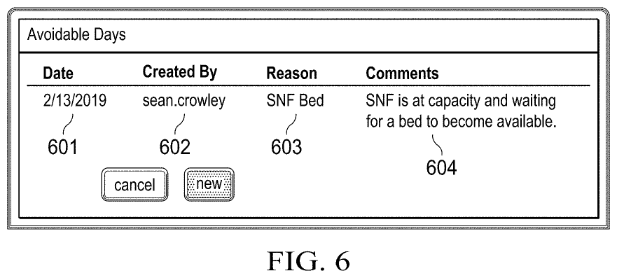

Once the reason for the delay and the related comments have been entered at the Avoidable Days entry screen, the charge date, selected reason, and comments will be visible in the entry fields, as seen in FIG. 5C. When all data entry has been completed, clicking the save button 506 will save the entries in the Avoidable Days module. After the data is saved in the Avoidable Days entry screen, the user is returned to the Avoidable Days display screen, where details from the recent entry are displayed. As seen in FIG. 6, the details of the entry are: the date 601 of the Avoidable Day; the name of the provider who created the entry 602; the selected reason 603; and the comments 604. Separate entries are made for each Avoidable Day incident.

When data has been captured by the claimed system and method, the data can be analyzed by date, patient, provider and/or reason for delay. Avoidable Days reports can be generated on-demand by a user or generated automatically on a set scheduled basis, such as, daily, weekly, monthly, etc. Reports can be generated by operators or managers for the facility, as well as by the physicians. The results can be reviewed and reported to the management team. Analysis of the each of the elements provides data that can be used to remediate the causes of the avoidable days in order to reduce or eliminate the Avoidable Days and decrease revenue losses associated with those days.

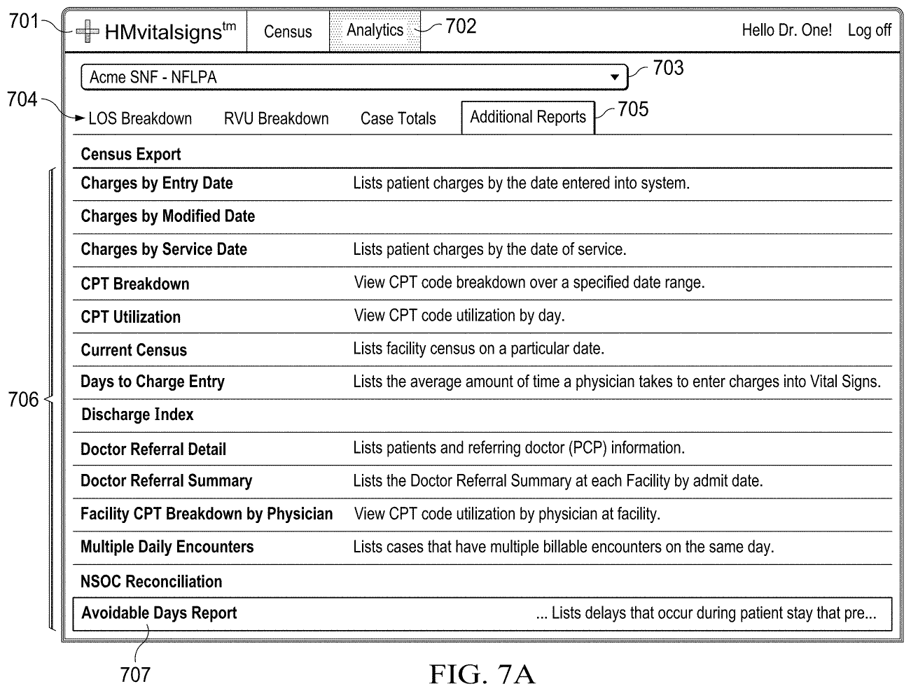

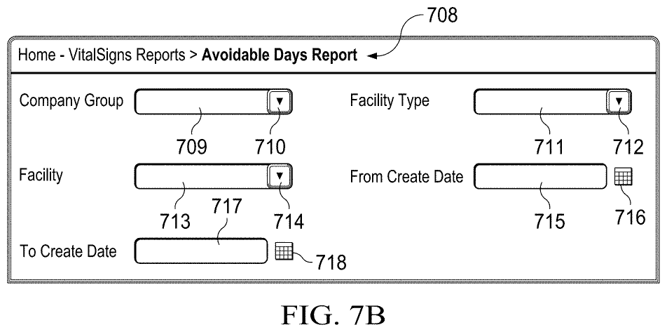

To generate a report from data entered at the Avoidable Days entry screen, the user accesses the analytics section of the Hospital Information System. FIG. 7A shows a dashboard for a Hospital Information System with an analytics tab 702 shown on the header bar 701. Selecting the analytics tab 702 displays available reporting and list options. A facility navigation bar 703 allows the user to select a specific facility for report generation. A facility can be selected from a dropdown menu or typing in the window will auto-fill options for the letters input in the window. An analytics navigation bar 703 has several tabs available for specific types of reports. To access the Avoidable Days reporting function, the user selects the Additional Reports tab 705 on the navigation bar. When the Additional Reports tab is selected, a listing of additional reports 706 is displayed and the user selects the Avoidable Days Report 707 option.

When Avoidable Days Report 707 is selected, an Avoidable Days Report entry screen (FIG. 7B) shows fields where the user can select the desired parameters for the report. Navigation bar 708 will direct the User back to a previous screen. Selectable parameters include Company Group, Facility Type, Facility, From Creation Date and To Creation Date. Users can select a Company Group by manually entering text in the entry field 709 or selecting the Company Group dropdown arrow 710 to display a list of selectable options. Users can select a Facility Type by manually entering text in the Facility Type entry field 711 or selecting the Facility Type from the dropdown arrow 712 to display a list of selectable options. Users can also select a Facility by name by manually entering text in the Facility entry field 713 or selecting the Facility name from the dropdown arrow 714 to display a list of selectable options. Text entry fields (709, 711, 713) will also auto-fill with selectable matching text. A starting date range for the report is entered in the From Creation Date entry field 715 and an ending date is entered in the To Created Date entry field 717. Starting and ending dates can also be selected from pop-up calendars (716, 718) located beside the date entry field. Selecting a date range generates a listing of Avoidable Days for the selected facility over the date range entered. The Avoidable Days report is populated by data entered at the Avoidable Days entry along with patient data, such as Medical Records Number (MRN) and Physicians Consulting Network (PCN), from the patient's Quality Panel. Once the report is rendered, the user can save the report, or export the data as a text file, CSV file, or XML data file to a portable document format (PDF), spreadsheet format, or word processing format. Once the report is exported, the data can be managed or sorted by various data properties, such as by date, patient, creator, doctor, or reason for the discharge delay.

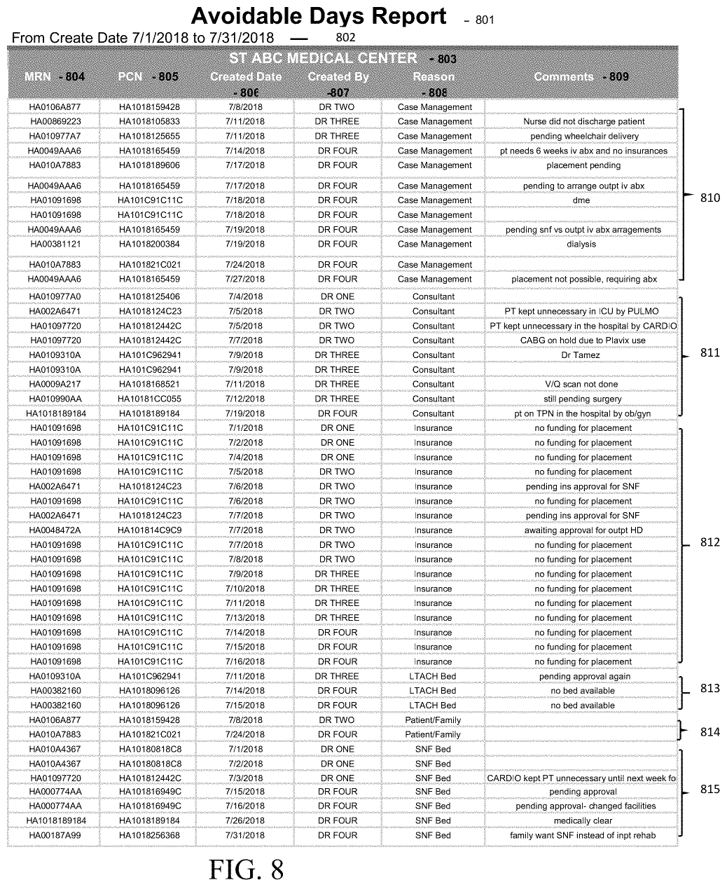

FIG. 8 shows an example of an Avoidable Days report 801 for the date range 802 selected for the chosen facility 803. The example spreadsheet report shows the Avoidable Days recorded over a one month span (Jul. 1, 2018 to Jul. 31, 2018) for St. ABC Medical Facility. As seen in the header bar, the data fields displayed for each entry include the Medical Records Number (MRN) 804 for the patient, the Physicians Consulting Network code (PCN) 805 associated with the case, the date for the Avoidable Day 806, the creator of the entry 807, the reason 808 for the Avoidable Day, and comments 809 related to the Avoidable Day entry. In FIG. 8, the data is sorted by the reason for the discharge delay 808. As seen in FIG. 8, Case Management 810 was the reason entered for 12 Avoidable Day entries, Consultant 811 was the reason entered for 9 days, Insurance 812 was the reason entered for 17 days, LTACH Bed 813 was the reason entered for 3 days, Patient/Family 814 was the reason entered for 2 days, SNF Bed 813 was the reason entered for 7 days, and there were no entries for Surgical delays for the selected date range.

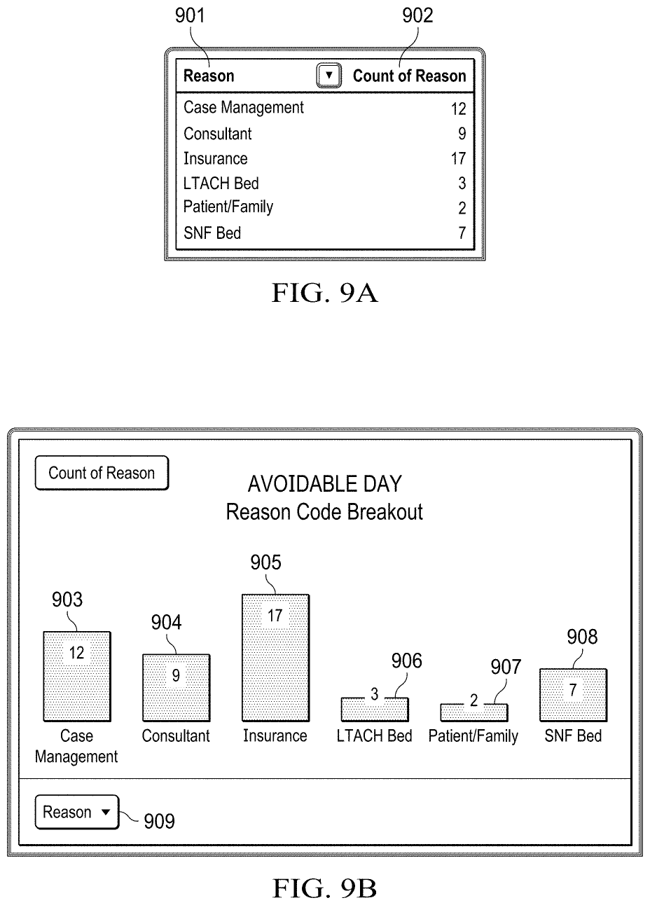

Report results can be displayed in several formats, including tables and charts. FIG. 9A shows a numeric table with columns for Reasons entered for Avoidable Days and the Count of those Reasons over the selected time frame. The Reasons column 901 lists the reasons as selected in the Avoidable Days entry screen and the number of days in the column for Count of Reasons 902 for the date range of Jul. 1, 2018 to Jul. 31, 2018, corresponds to data in the FIG. 8 Avoidable Days Report. In the FIG. 9A table, it can be seen that for the selected time period, Insurance was selected most often as an Avoidable Days reason with 17 occurrences over the selected month of July 2018. Patient/Family was selected least as a reason for Avoidable Days with only 2 occurrences over the selected month. Surgery was not entered as reason during the July 2018 date range, therefore no occurrences of Surgery appear on the table.

FIG. 9B shows the same data from the FIG. 9A table displayed as a visual representation in a bar chart of the Avoidable Day Reason Code Breakout. The Count of Reason bars are sized based on the number of occurrences for each reason selected, with larger bars indicating more occurrences, and shows Case Management 903 occurs 12 times, Consultant 904 occurs 9 times, Insurance 905 occurs 17 times, LTACH Bed 906 occurs 3 times, Patient/Family 907 occurs 2 times, and SNF Bed 908 occurs 7 times, in agreement with the counts presented in the FIG. 8 and the FIG. 9A table. In the bar chart format of FIG. 9B, the largest bar (Insurance 905) is easy to compare visually with the smallest bar (Patient/Family 907), and the numeric counts are also included for each reason bar.

If the Avoidable Days report is exported and rendered in a spreadsheet-type format, the data can be manipulated to provide topic specific reports, for example, by patient, reason or physician. Topic specific reports provide value to the management team by allowing additional analysis of the Avoidable Days data. Analysis of these types of topic specific reports may help a facility's management to understand why avoidable days occur in their facility and what steps might be taken to reduce those days. Analysis of the types of topic specific reports help a facility's management team to better understand the causes of avoidable days occurring in their facility and what steps might be taken to reduce those days. Analyzing by patient would show that the bulk of the Avoidable Days for the examined period are attributed to a single patient and the reason is coded as Insurance because there was no funding for placement of the patient, and this one situation accounts for about one third of the Avoidable Days entries over the date range of July 1-July 31.

The Avoidable Days Reports and examples described herein are only a tiny sampling of the Avoidable Days issues faced by most medical facilities. There are more than 6000 hospitals in the United States, with most of these hospitals having over 100 beds, and some of the larger medical centers having over 900 beds, which yields more than 900,000 staffed beds overall. A large medical center can easily have over 100 un-reimbursed Avoidable Days each month and when calculated over the entire year, the revenue losses skyrocket. When considered for all of the estimated over 6000 hospitals in the country, the revenue losses attributable to Avoidable Days are enormous and present an extreme financial burden to the hospitals, impeding their ability to provide the desired level of excellence in medical care.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention.

1. A system for determining and reporting an avoidable days discharge delay event and a first facility causation for a patient that is ready for discharge from a first facility, said first facility causation is associated with said first facility operations or personnel and not related to the patient's clinical data or the patient's condition, comprising:
a hardware data processor coupled to a plurality of non-transitory storage devices and one or more input/output ports coupled to one or more input/output devices, said hardware data processor executes an avoidable days discharge delay subprogram,
said non-transitory storage devices maintains a first set of facility data related to the first facility operations and personnel, and a first set of patient data including patient identification, patient treatment information, facility identification, and a discharge date for the patient,
said input/output ports provides for management of data properties related to said first set of facility data stored in said non-transitory storage devices and said first set of patient data related to the patient admitted to first facility, said management of data properties includes conversion of patient data into a standardized data format and storage of patient data in said standardized data format;
upon modification of the patient's discharge date data to a later date for the patient that is ready for discharge from the first facility, said hardware data processor executes an avoidable days discharge delay subprogram to determine one or more first facility causations associated with the first facility operations or personnel and not related to the patient's clinical data or the patient's condition, said avoidable days discharge delay subprogram having the following functionalities:
(a) accesses the first set of facility data stored in said non-transitory storage devices for the first facility where the patient is admitted, including data relating to the first facility operations and personnel,
(b) analyzes said first set of facility data to identify the first facility causation correlated to the modification in the discharge date for the patient that is ready for discharge from the first facility, said analysis includes a review of said first set of facility data associated with the first facility operations and personnel without consideration of the patient's clinical data or the patient's condition,
(c) determines if said first facility causation constitutes an avoidable days discharge delay event from the analysis of said first set of facility data without consideration of the patient's clinical data and the patient's condition,
(d) generates one or more reports identifying the avoidable days discharge delay event and the first facility causation with a description of the reason for the discharge delay associated with first facility where the patient is admitted, said one or more reports being stored in said non-transitory memory storage; and,
(e) transmitting and exporting through said input/output ports coupled to said hardware data processor said one or more reports identifying the avoidable days discharge delay event and the first facility causation to a predetermined group of personnel.

2. The system of claim 1 wherein the first facility causation for the delay in the discharge date is input based on a selection of causes from a menu.

3. The system of claim 1 wherein the first facility causation for the delay in the discharge date is input based on a manual entry of a cause.

4. The system of claim 1 wherein said avoidable days discharge delay subprogram provides an on-screen alert under predetermined circumstances.

5. The system of claim 1 wherein said avoidable days discharge delay subprogram receives user input for the analysis of causation of the delay in the discharge date for the patient.

6. The system of claim 1 wherein the patient data includes one or more comments relating to the reasons for any delay in the discharge date.

7. The system of claim 1 wherein reports are generated using a starting and ending date range.

8. The system of claim 7 wherein said starting and ending date range is entered manually or selected from a pop-up calendar with selectable dates.

9. The system of claim 1 wherein said first set of facility data includes a facility name, facility type, and facility group related to the first facility.

10. The system of claim 9 wherein said facility name, facility type or facility group can be entered manually or selected from one or more lists of selectable facility names, facility types or facility groups.

11. A system for determining and reporting an avoidable days discharge delay event and a first facility causation for a patient that is ready for discharge from a first facility, said first facility causation is associated with said first facility operations or personnel and not related to the patient's clinical data or the patient's condition, comprising:
a hardware data processor coupled to a plurality of non-transitory storage devices and one or more input/output ports coupled to one or more input/output devices, said hardware data processor executes an avoidable days discharge delay subprogram,
said non-transitory storage devices maintains a first set of facility data related to the first facility operations and personnel, and a first set of patient data including patient identification, patient treatment information, facility identification, and a discharge date for the patient,
said input/output ports provides for management of data properties related to said first set of facility data stored in said non-transitory storage devices and patient data related to the patient admitted to first facility,
upon modification of the patient's discharge date data to a later date for the patient that is ready for discharge from the first facility, said hardware data processor executes an avoidable days discharge delay subprogram to determine one or more first facility causations associated with the first facility operations or personnel and not related to the patient's clinical data or the patient's condition, said avoidable days discharge delay subprogram having the following functionalities:
(a) accesses the first set of facility data stored in said non-transitory storage devices for the first facility where the patient is admitted, including data relating to the first facility operations and personnel,
(b) analyzes said first set of facility data to identify the first facility causation correlated to the modification in the discharge date for the patient that is ready for discharge from the first facility, said analysis includes a review of said first set of facility data associated with the first facility operations and personnel without consideration of the patient's clinical data or the patient's condition,
(c) determines if said first facility causation constitutes an avoidable days discharge delay event from the analysis of said first set of facility data without consideration of the patient's clinical data and the patient's condition,
(d) generates one or more reports identifying the avoidable days discharge delay event and the first facility causation with a description of the reason for the discharge delay associated with first facility where the patient is admitted, said one or more reports being stored in said non-transitory memory storage; and,
(e) provides an on-screen alert related to the avoidable days discharge delay event and the first facility causation with a description of the reason for the discharge delay that is associated with first facility where the patient is admitted; and,
(f) transmitting and exporting through said input/output ports coupled to said hardware data processor said one or more reports identifying the avoidable days discharge delay event and the first facility causation to a predetermined group of personnel.

12. The system of claim 11 wherein the first facility causation for the delay in the discharge date is input based on a selection of causes from a menu.

13. The system of claim 11 wherein the first facility causation for the delay in the discharge date is input based on a manual entry of a cause.

14. The system of claim 11 wherein said avoidable days discharge delay subprogram provides an on-screen alert under predetermined circumstances.

15. The system of claim 11 wherein said avoidable days discharge delay subprogram receives user input for the analysis of causation of the delay in the discharge date for the patient.

16. The system of claim 11 wherein the patient data includes one or more comments relating to the reasons for any delay in the discharge date.

17. The system of claim 11 wherein reports are generated using a starting and ending date range.

18. The system of claim 17 wherein said starting and ending date range is entered manually or selected from a pop-up calendar with selectable dates.

19. The system of claim 11 wherein said first set of facility data includes a facility name, facility type, and facility group related to the first facility.

20. The system of claim 19 wherein said facility name, facility type or facility group can be entered manually or selected from one or more lists of selectable facility names, facility types or facility groups.

21. A system for determining and reporting an avoidable days discharge delay event and a first facility causation for a patient that is ready for discharge from a first facility, said first facility causation is associated with said first facility operations or personnel and not related to the patient's clinical data or the patient's condition, comprising:
a hardware data processor coupled to a plurality of non-transitory storage devices and one or more input/output ports coupled to one or more input/output devices, said hardware data processor executes an avoidable days discharge delay subprogram,
said non-transitory storage devices maintains a first set of facility data related to the first facility operations and personnel, and patient data including patient identification, patient treatment information, facility identification, and a discharge date for the patient,
said input/output ports provides for management of data properties related to said first set of facility data stored in said non-transitory storage devices and patient data related to the patient admitted to first facility,
upon modification of the patient's discharge date data to a later date for the patient that is ready for discharge from the first facility, said hardware data processor executes an avoidable days discharge delay subprogram to determine one or more first facility causations associated with the first facility operations or personnel and not related to the patient's clinical data or the patient's condition, said avoidable days discharge delay subprogram having the following functionalities:
(a) accesses the first set of facility data stored in said non-transitory storage devices for the first facility where the patient is admitted, including data relating to the first facility operations and personnel,
(b) analyzes said first set of facility data to identify the first facility causation correlated to the modification in the discharge date for the patient that is ready for discharge from the first facility, said analysis includes a review of said first set of facility data associated with the first facility operations and personnel without consideration of the patient's clinical data or the patient's condition,
(c) determines if said first facility causation constitutes an avoidable days discharge delay event from the analysis of said first set of facility data without consideration of the patient's clinical data and the patient's condition,
(d) generates one or more reports identifying the avoidable days discharge delay event and the first facility causation with a description of the reason for the discharge delay associated with first facility where the patient is admitted, said one or more reports being stored in said non-transitory memory storage; and,
(e) transmitting and exporting through said input/output ports coupled to said hardware data processor said one or more reports identifying the avoidable days discharge delay event and the first facility causation to a predetermined group of personnel.

22. The system of claim 21 wherein the first facility causation for the delay in the discharge date is input based on a selection of causes from a menu.

23. The system of claim 21 wherein the first facility causation for the delay in the discharge date is input based on a manual entry of a cause.

24. The system of claim 21 wherein said avoidable days discharge delay subprogram provides an on-screen alert under predetermined circumstances.

25. The system of claim 21 wherein said avoidable days discharge delay subprogram receives user input for the analysis of causation of the delay in the discharge date for the patient.

26. The system of claim 21 wherein the patient data includes one or more comments relating to the reasons for any delay in the discharge date.

27. The system of claim 21 wherein reports are generated using a starting and ending date range.

28. The system of claim 27 wherein said starting and ending date range is entered manually or selected from a pop-up calendar with selectable dates.

29. The system of claim 21 wherein said first set of facility data includes a facility name, facility type, and facility group related to the first facility.

30. The system of claim 29 wherein said facility name, facility type or facility group can be entered manually or selected from one or more lists of selectable facility names, facility types or facility groups.
